(19) United States
(12) Patent Application Publication
(10) Pub. No.: US 2016/0144115 A1
(43) Pub. Date: May 26, 2016

(54) MEDICATION CONTROL METHOD, MEDICATION CONTROL PROGRAM, AND MEDICATION SYSTEM

(71) Applicant: SEIKO EPSON CORPORATION, Tokyo (JP)

(72) Inventor: Yasuhiro MOMOSE, Matsumoto-shi (JP)

(21) Appl. No.: 14/948,051

(22) Filed: Nov. 20, 2015

(30) Foreign Application Priority Data
Nov. 21, 2014 (JP) 2014-236288

(51) Int. Cl. A61M 5/172; G06F 19/00; G05B 15/02

(52) U.S. Cl. CPC A61M 5/1723; G05B 15/02; G06F 19/3468; A61M 2205/3553; A61M 2205/52; A61M 2205/50

(57) ABSTRACT

A medication control method includes: a medication instruction step in which an instruction to perform medication to a living body is given; a biological information gathering instruction step in which an instruction to perform an operation of detecting biological information that can change due to the medication as a factor and gathering the biological information as monitoring information is given; and an instruction step in which one of the medication instruction step and the biological information gathering instruction step is inhibited or postponed if a period of detection of the biological information carried out according to the biological information gathering instruction step and a period during which the medication carried out according to the medication instruction step influences the detection of the biological information coincide with each other.

### BACKGROUND

1. Technical Field

The present invention relates to a medication control method, a medication control program, and a medication system.

2. Related Art

A medicine injection device is widely known, as disclosed in JP-A-2004-283378. The medicine injection device has an indwelling needle. Insulin is administered into a patient's body from the indwelling needle. Prior to the administration, the blood glucose level or blood sugar concentration is measured. A sensor fixed on the lateral side of the indwelling needle is used for the measurement of the concentration.

The insulin administered from the indwelling needle facilitates consumption of glucose within cells. As a result, the blood glucose level falls locally at the insulin injection site at the time of injection. Therefore, if the site where the blood glucose level is measured and the insulin injection site are close to each other, the blood glucose level falls locally at the measurement site and the blood glucose level cannot be measured correctly. This may pose the risk that correct administration of insulin cannot be carried out.

### SUMMARY

An advantage of at least one aspect of the invention is to provide a medication control method that contributes to gathering correct biological information even if the medication site and the site where biological information is detected are close to each other.

(1) An aspect of the invention relates to a medication control method including: a medication instruction step in which an instruction to perform medication to a living body is given; a biological information gathering instruction step in which an instruction to perform an operation of detecting biological information that can change due to the medication as a factor and gathering the biological information as monitoring information is given; and an instruction step in which one of the medication instruction step and the biological information gathering instruction step is inhibited or postponed if a period of detection of the biological information carried out according to the biological information gathering instruction step and a period during which the medication carried out according to the medication instruction step influences the detection of the biological information coincide with each other.

When an instruction to perform medication is given, biological information changes toward a normal value locally at the medication site in the living body. When an instruction to perform an operation of gathering biological information is given, the biological information is gathered at the detection site in the living body. In this embodiment, one of the medication instruction step and the biological information gathering instruction step is inhibited or postponed, even if the period of detection of the biological information and the period during which the medication influences the detection of the biological information coincide with each other. Therefore, the local change in the biological information is not gathered as the monitoring information. Thus, even if the medication site and the site where the biological information is detected are close to each other, the medication can be carried out on the basis of correct biological information.

(2) In the medication control method, a period during which an interrupt instruction is given may be defined as the period during which the medication carried out according to the medication instruction step has influence, and a start of the period during which the interrupt instruction is given with respect to a medication start timing and an end of the period during which the interrupt instruction is given with respect to a medication end timing may be set. The period of the interrupt instruction is thus adjusted. The coincidence of the medication instruction step and the biological information gathering instruction step is securely avoided. According to conditions that influence the interrupt instruction, a time indicating how long the detection is to be inhibited prior to the start of the medication can be set and a time indicating how long the inhibition period is to last following the medication can be set. The medication can be carried out on the basis of correct biological information. Moreover, in addition to these effects, the shift of the biological information gathering from the timing of the medication can be reduced to the minimum.

(3) In the instruction step, the detection of the biological information may be inhibited. If the detection of the biological information is inhibited, the local change in the biological information is not gathered as the monitoring information. Therefore, the medication can be carried out on the basis of correct biological information even if the medication site and the site where the biological information is detected are close to each other. Moreover, in addition to these effects, no change is made to the timing of the medication.

(4) In the instruction step, gathering the biological information detected according to the biological information gathering instruction step, as the monitoring information, may be inhibited. The gathering of the monitoring information is inhibited in the instruction step even if the biological information is detected according to the biological information gathering instruction step. The local change in the biological information is not gathered as the monitoring information. Therefore, the medication can be carried out on the basis of correct biological information even if the medication site and the site where the biological information is detected are close to each other. Moreover, in addition to these effects, no change is made to the timing of the biological information gathering and the timing of the medication.

(5) In the interrupt instruction step, the medication instruction step may be postponed until the period of detection of the biological information ends. Even if the instruction to perform the detection of the biological information is given repeatedly according to the setting, the medication instruction step does not coincide with the period of detection of the biological information. Thus, the medication is avoided at the time of gathering the biological information. The medication does not influence the detection of the biological information. The local change in the biological information is not gathered as the monitoring information. Therefore, the medication can be carried out on the basis of correct biological information even if the medication site and the site where the biological information is detected are close to each other. Moreover, in addition to these effects, no change is made to the operation of detecting the biological information.

(6) In the instruction step, the medication instruction step may be postponed until transfer of the biological information that is detected is received, and the biological information gathering instruction step may be postponed until after the transfer of the biological information is received and until after it is confirmed that the medication instruction step is absent or until after the lapse of the period during which the medication carried out according to the medication instruction step has influence. Since the medication instruction step is postponed until the transfer of the biological information is received, the coincidence of the medication instruction step and the biological information gathering instruction step is securely avoided. After that, if it is confirmed that the medication instruction step is absent or if the period during which the medication has influence passes after the reception of the transfer of the biological information, the coincidence of the medication instruction step and the biological information gathering instruction step is avoided. The local change in the biological information is not gathered as the monitoring information. Therefore, the medication can be carried out on the basis of correct biological information even if the medication site and the site where the biological information is detected are close to each other. Moreover, in addition to these effects, the coincidence of the medication instruction step and the biological information gathering instruction step can be avoided even if the timing of the medication is set arbitrarily.

(7) Another aspect of the invention relates to a medication control program causing a computer to carry out: a medication instruction procedure in which an instruction to perform medication to a living body is given; a biological information gathering instruction procedure in which an instruction to perform an operation of detecting biological information that can change due to the medication as a factor and gathering the biological information as monitoring information is given; and an instruction procedure in which one of the medication instruction procedure and the biological information gathering instruction procedure is inhibited or postponed if a period of detection of the biological information carried out according to the biological information gathering instruction procedure and a period during which the medication carried out according to the medication instruction procedure influences the detection of the biological information coincide with each other.

(8) Still another aspect of the invention relates to a medication system including: a medication unit which performs medication to a living body; a biological information gathering unit which detects biological information that can change due to the medication as a factor, and which gathers the biological information as monitoring information; and a control unit which gives an instruction to perform the medication and to perform an operation of gathering the monitoring information, and carries out processing to inhibit or postpone one of the instruction to perform the medication and the detection of the biological information if a period of detection of the biological information and a period during which the medication influences the detection of the biological information coincide with each other.

### BRIEF DESCRIPTION OF THE DRAWINGS

The invention will be described with reference to the accompanying drawings, wherein like numbers reference like elements.

FIG. 1 is an exterior view schematically showing the overall configuration of a medication system according an embodiment.

FIG. 2 is a block diagram of the medication system.

FIG. 3 is a chart schematically showing the operation of a medication system according a first embodiment.

FIG. 4 is a diagram of subcutaneous tissues, schematically showing medication and detection of blood glucose level.

FIG. 5 is a chart schematically showing the operation of a medication system according a second embodiment.

FIG. 6 is a chart schematically showing the operation of a medication system according a third embodiment.

FIG. 7 is a chart schematically showing the operation of a medication system according a fourth embodiment.

### DESCRIPTION OF EXEMPLARY EMBODIMENTS

Hereinafter, an embodiment of the invention will be described with reference to the accompanying drawings. The embodiment described below should not unduly limit the contents of the invention described in the appended claims. Not all the configurations described in the embodiment are essential as the solutions according to the invention.

### (1) Overall Configuration of Medication System

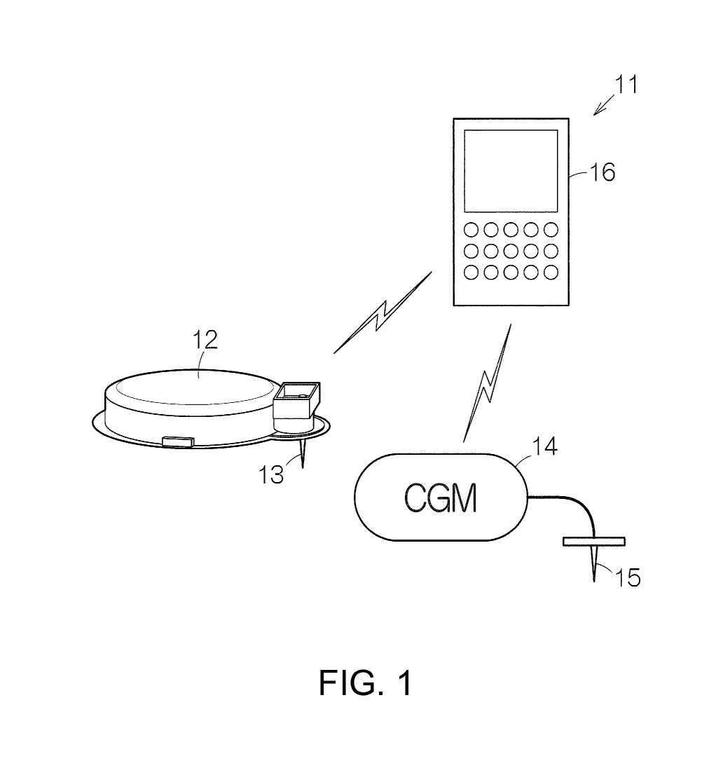

FIG. 1 schematically shows the overall configuration of a medication system 11 according to an embodiment. The medication system 11 has an insulin pump (medication unit) 12. The insulin pump 12 has an indwelling needle 13. The indwelling needle 13 is inserted in a living body, for example, a human body. The point of the indwelling needle 13 reaches the interstitial fluid in a subcutaneous tissue, for example. As the insulin pump 12 is actuated, medication is carried out toward the subcutaneous tissue. By the medication, insulin is supplied to the interstitial fluid.

The operation of the insulin pump 12 includes basal injection and bolus injection. In basal injection, insulin is continuously injected into the living body from the indwelling needle 13. The insulin is administered without interruption as long as it continues cyclically at an interval of 5 minutes or 15 minutes. Meanwhile, in bolus injection, insulin is injected into the living body from the indwelling needle 13 in a spot-like manner at a specific timing. The insulin is administered to the living body cyclically over several minutes, for example, according to a predetermined time point or the designation by the user.

The medication system 11 has a blood glucose meter (biological information gathering unit) 14. The blood glucose meter 14 has a pair of electrodes 15, for example. The electrodes 15 are inserted, for example, into the living body. The tips of the electrodes 15 are immersed, for example, in the interstitial fluid in a subcutaneous tissue. The blood glucose meter 14 detects the blood glucose level, that is, glucose concentration (biological information) in the interstitial fluid. As the blood glucose meter 14, for example, a CGM (continuous glucose sensor) can be used. The blood glucose meter 14 outputs data of blood glucose level periodically at an interval of 3 minutes or 5 minutes.

The medication system 11 has a controller (control unit) 16. The controller 16 controls the operation of the insulin pump 12. To control the operation, the controller 16 gathers the data of blood glucose level as monitoring information from the blood glucose meter 14. If an excessive fall in the blood glucose level is expected from the data of blood glucose level, the controller 16 issues a warning to the user or gives an instruction to stop the medication. As the controller 16, for example, a portable laptop personal computer, smartphone, tablet personal computer, and other similar devices can be used.

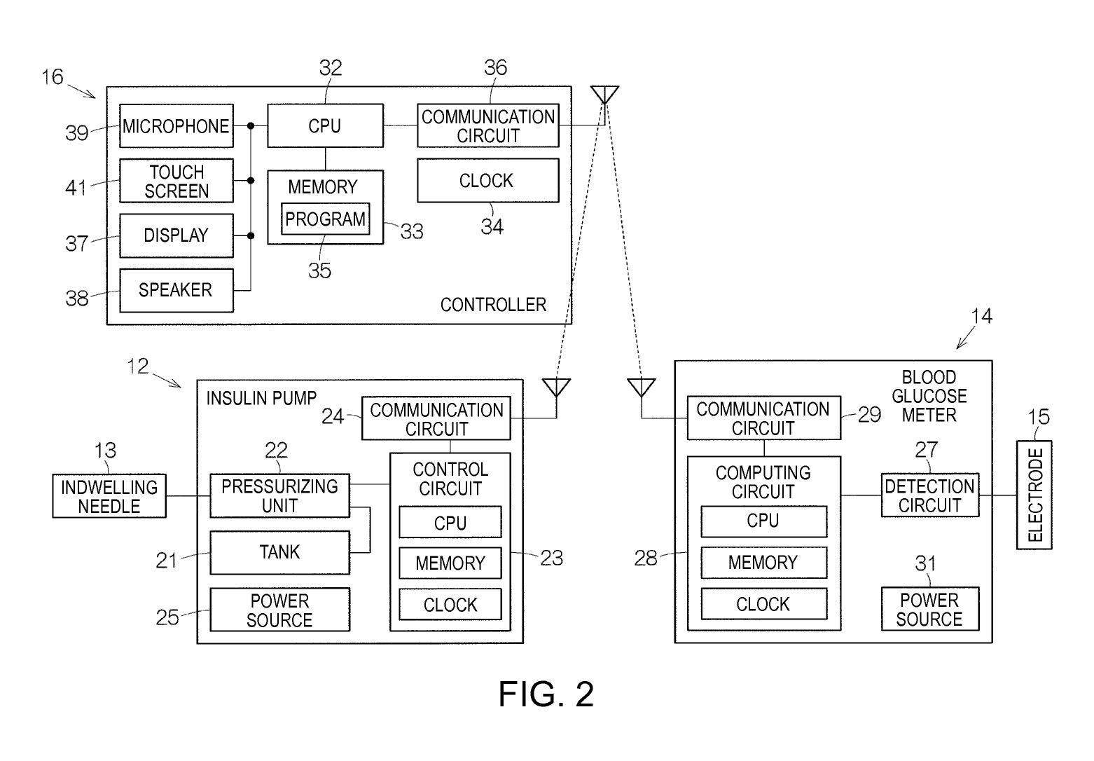

As shown in FIG. 2, the insulin pump 12 has a tank 21. Insulin is stored in the tank 21. The tank 21 can be formed, for example, as a cartridge-type tank. The insulin pump 12 can be refilled with insulin when the cartridge is replaced.

The insulin pump 12 has a pressurizing unit 22. The tank 21 is connected to the pressurizing unit 22. The pressurizing unit 22 acts to cause the insulin to be discharged from the tank 21. The discharged insulin is supplied to the indwelling needle 13. The pressurizing unit 22 has a motor, for example. A pressure is generated in the pressurizing unit 22 in response to the drive force of the motor. The generated pressure acts to cause the insulin to flow from the tank 21 to the indwelling needle 13. The motor can be made up of a piezoelectric motor, for example. The piezoelectric motor generates a drive force through the action of a piezoelectric element.

The insulin pump 12 has a control circuit 23. The control circuit 23 is connected to the pressurizing unit 22. The control circuit 23 controls the operation of the pressurizing unit 22. The control circuit 23 can be made up of, for example, a CPU (central processing unit), a memory, and a clock circuit. The CPU operates according to a program stored in the memory. The operation of the CPU is synchronized with a clock generated by the clock circuit. Here, the operation of the pressurizing unit 22 is repeated, for example, on a cycle R.

The insulin pump 12 has a communication circuit 24. The communication circuit 24 is connected to the control circuit 23. The communication circuit 24 can receive a control signal from outside, for example, via wireless communication. The control circuit 23 can operate according to a control signal received from the communication circuit 24. The communication circuit 24 may utilize Bluetooth (trademark registered), for example. The communication circuit 24 may also transmit signals such as error message and status message to outside.

The insulin pump 12 has a power source 25. The power source 25 is connected to the control circuit 23, the pressurizing unit 22 and the communication circuit 24. The power source 25 can be made up of a battery, for example. The control circuit 23, the pressurizing unit 22 and the communication circuit 24 operate according to the power supplied from the power source 25.

The blood glucose meter 14 has a detection circuit 27. The detection circuit 27 is connected to the electrodes 15. The detection circuit 27 measures a current generated between the electrodes 15. Here, to give an example of the principle of the detection circuit 27, when glucose in the interstitial fluid in the subcutaneous tissue passes through a glucose oxidase enzyme membrane, gluconic acid and hydrogen peroxide are generated from the glucose and enzyme. The hydrogen peroxide thus generated is further resolved into water and oxygen on the electrode surfaces. At that point, a reaction current is generated between the electrodes 15. This current is proportionate to the amount of hydrogen peroxide. Since the amount of hydrogen peroxide is proportionate to the glucose concentration in the interstitial fluid, the reaction current value that is actually measured can be converted to the glucose concentration in the interstitial fluid.

The blood glucose meter 14 has a computing circuit 28. The computing circuit 28 is connected to the detection circuit 27. The computing circuit 28 acquires the result of the detection by the detection circuit 27 at a time interval of 5 seconds or 10 seconds, for example. The computing circuit 28 calculates an average value from the results of the detection by the detection circuit 27 on a cycle P of 5 minutes or 10 minutes, and outputs the average value as data of blood glucose level. The computing circuit 28 can be made up of a CPU (central processing unit), a memory, and a clock circuit, for example. The CPU operates according to a program stored in the memory. The operation of the CPU is synchronized with a clock generated by the clock circuit.

The blood glucose meter 14 has a communication circuit 29. The communication circuit 29 is connected to the computing circuit 28. The communication circuit 29 can transmit the data of blood glucose level to outside, for example, via wireless communication. Similarly, the communication circuit 29 can receive a control signal from outside via wireless communication. The computing circuit 28 can operate according to the control signal received by the communication circuit 29. The control signal includes an inhibiting signal that inhibits the detection of blood glucose level. The communication circuit 29 may utilize Bluetooth (trademark registered), for example.

The blood glucose meter 14 has a power source 31. The power source 31 is connected to the computing circuit 28, the detection circuit 27 and the communication circuit 29. The power source 31 can be made up of a battery, for example. The computing circuit 28, the detection circuit 27 and the communication circuit 29 operate according to the power supplied from the power source 31.

The controller 16 has a CPU (central processing unit) 32. A memory 33 is connected to the CPU 32. The CPU 32 operates according to a program stored in the memory 33. The operation of the CPU 32 may be synchronized, for example, with a clock generated by a clock circuit 34. A medication control program 35, for example, is stored in the memory 33. The medication control program 35 may be downloaded from a website, for example, and then stored in the memory 33.

The controller 16 has a communication circuit 36. The communication circuit 36 is connected to the CPU 32. The CPU 32 can transmit a control signal to the communication circuit 24 of the insulin pump 12, for example, via wireless communication. Similarly, the CPU 32 can receive the data of blood glucose level from the communication circuit 29 of the blood glucose meter 14 and transmit a control signal to the communication circuit 29 of the blood glucose meter 14, via wireless communication.

The controller 16 may have output devices such as a display 37 and a speaker 38, and input devices such as a microphone 39 and a touch screen panel 41. The controller 16 can issue a warning to the user via the display 37 and the speaker 38. The user can input instructions, settings and data to the controller 16 via the microphone 39 and the touch screen panel 41.

### (2) Operation of Medication System According to First Embodiment

Next, the operation of the medication system 11 will be described. As the controller 16 executes the medication control program 35, the medication control method is carried out. When carrying out the medication control method, the insulin pump 12 and the blood glucose meter 14 are associated with the controller 16. Here, the insulin pump 12 and the blood glucose meter 14 are wirelessly connected to the controller 16. A wireless channel between the controller 16, and the insulin pump 12 and the blood glucose meter 14, is established via Bluetooth. The insulin pump 12 and the blood glucose meter 14 are registered with the controller 16.

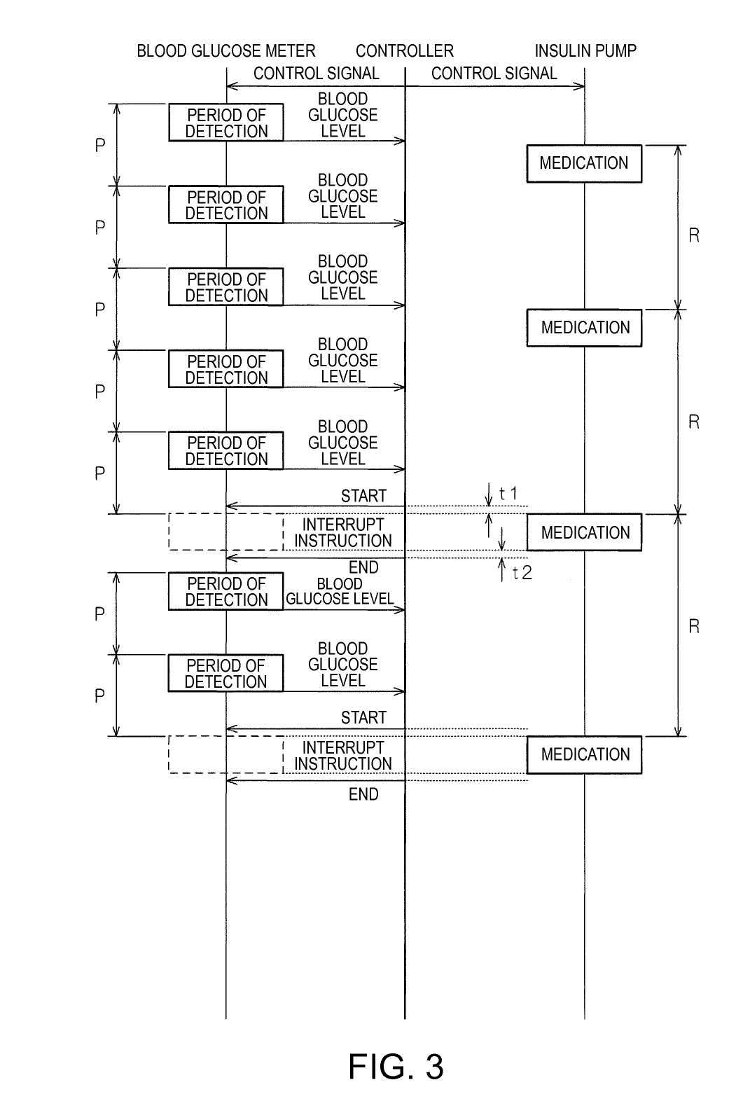

When the power source 31 of the blood glucose meter is switched on, the blood glucose meter 14 can start measuring the blood glucose level. As shown in FIG. 3, the controller 16 can generate a control signal for the blood glucose meter 14. Based on the control signal, the controller 16 instructs the blood glucose meter 14 to perform an operation of detecting the blood glucose level and gathering the blood glucose level as monitoring information. Thus, the biological information gathering instruction step is realized.

The blood glucose meter 14 supplies the data of blood glucose level to the controller 16. The blood glucose meter 14 carries out measurement at a unique timing according to the clock generated by the internal clock circuit. That is, the blood glucose meter 14 repeats the operation of detecting and gathering the blood glucose level as monitoring information on the cycle P. At this time, the controller 16 acquires a time stamp of the blood glucose meter 14. The time stamp may be incorporated in the data of blood glucose level. Thus, the controller 16 specifies the period of detection of the blood glucose level. The cycle P of detection of the blood glucose level may be registered in advance with the controller 16. Alternatively, the cycle P may be specified in response to the reception of a plurality of data.

The controller 16 generates a control signal for the insulin pump 12. An instruction to perform medication is specified in the control signal. When generating the control signal, the controller 16 specifies the cycle R of medication. The insulin pump 12 carries out medication in response to the reception of the control signal. The medication is repeated on the cycle R. The medication instruction step in which an instruction to perform medication to the living body is given is thus realized. The insulin is administered into the patient's body from the indwelling needle 13.

When the cycle R of medication is specified, the controller 16 specifies a period during which the medication influences the detection of the blood glucose level (hereinafter referred to as a “detection inhibiting period”) . The detection inhibiting period may be a period during which the administered insulin spreads in the interstitial fluid at the medication site, equalizing the local blood glucose level at the medication site to the blood glucose level in the surrounding interstitial fluid. Such a period may be customized on the basis of actual measurement with each individual or may be generalized on the basis of large sample data. As generally known, the blood glucose level changes due to the administration of insulin as a factor.

The controller 16 monitors the blood glucose level in the interstitial fluid in the subcutaneous tissue. If an excessive fall in the blood glucose level is predicted, the controller 16 stops the injection of insulin from the insulin pump 12. The controller 16 outputs a control signal to stop the operation of the pressurizing unit 22, to the insulin pump 12.

If the period of detection of the blood glucose level and the detection inhibiting period coincide with each other, the controller 16 generates an interrupt instruction to inhibit or postpone one of the medication instruction step and the biological information gathering instruction step Here, the detection of the blood glucose level is inhibited on the basis of the interrupt instruction. The period during which the interrupt instruction is issued is decided on the basis of the detection inhibiting period. In other words, the start of the period during which the interrupt instruction is issued with respect to the medication start time corresponds to the start of the detection inhibiting period, and the end of the period during which the interrupt instruction issued with respect to the medication end time corresponds to the end of the detection inhibiting period. With the start of the period, a time (t1) indicating how long the detection is to be inhibited prior to the start of the medication can be set, and with the end of the period, a time (t2) indicating how long the inhibition period is to last following the medication can be set. As described above, if the time stamp of the blood glucose meter 14 is specified in advance, the controller 16 may notify the blood glucose meter 14 of the start and end of the detection inhibiting period only when the period of detection of the blood glucose level and the detection inhibiting period coincide with each other. Also, the controller 16 may notify the blood glucose meter 14 of the start and end of the detection inhibiting period every time the controller 16 transmits the control signal for the insulin pump 12.

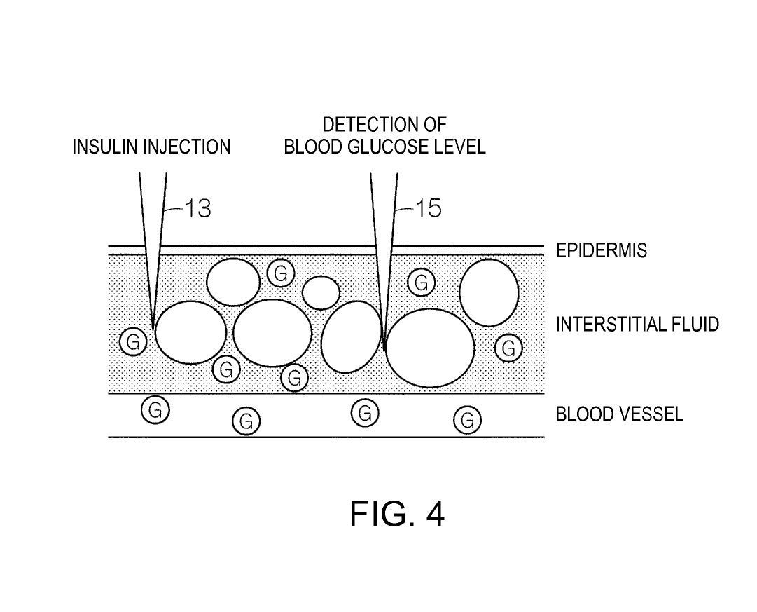

As shown in FIG. 4, when the insulin is supplied to the interstitial fluid in the subcutaneous tissue from the indwelling needle 13, the insulin permeates the blood vessel from the interstitial fluid and is then carried away. The insulin facilitates consumption of glucose G in cells. The blood glucose level successively falls. At the insulin injection site, the blood glucose level falls locally at the time of injection. The blood glucose level locally changes toward a normal value. At this time, in this embodiment, even if the period of detection of the blood glucose level and the detection inhibiting period coincide with each other, the biological information gathering instruction step is inhibited and therefore the local change in the blood glucose level is not gathered as the monitoring information. Therefore, even if the medication site and the site where the blood glucose level is detected are close to each other, the medication can be carried out on the basis of the correct blood glucose level.

The insulin is consumed with the consumption of the glucose G. Moreover, the insulin permeates the blood vessel and is carried away. Thus, the concentration of insulin successively falls at the medication site. Also, the glucose G percolates through the blood vessel into the interstitial fluid in the subcutaneous tissue. Consequently, the local fall in the blood glucose level at the medication site is resolved. As the local change in the blood glucose level is resolved, the detection inhibiting period ends. Thus, the blood glucose meter 14 resumes the detection of the blood glucose level.

In this embodiment, the period during which the interrupt instruction is issued is defined as the period during which the medication carried out according to the medication instruction step has influence, and the controller 16 sets the start of the period during which the interrupt instruction is issued with respect to the medication start time, and the end of the period during which the interrupt instruction is issued with respect to the medication end time. Thus, the period of the interrupt instruction is adjusted. The coincidence of the medication instruction step and the biological information gathering instruction step is securely avoided. The period of the interrupt instruction can be changed according to conditions that influence the interrupt instruction. For example, the controller 16 can also change the start and end according to the dosage.

### (3) Operation of Medication System According to Second Embodiment

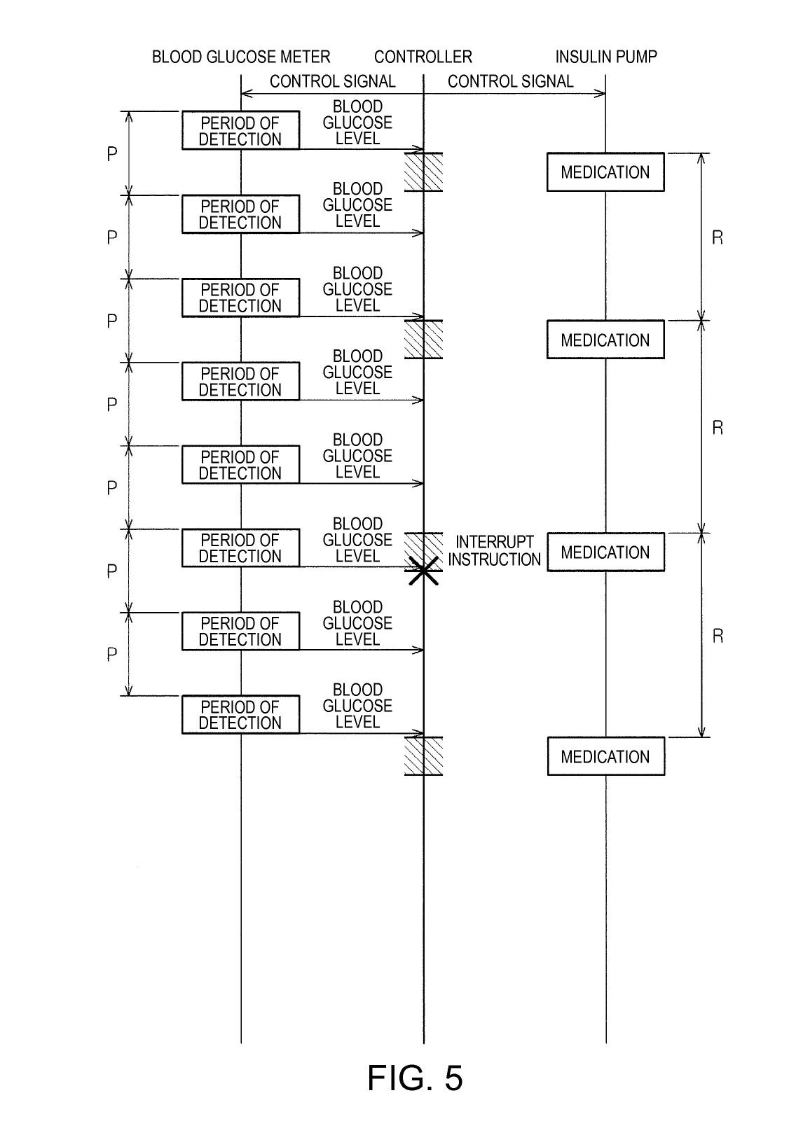

In a second embodiment, if the period of detection of the blood glucose level and the detection inhibiting period coincide with each other, the controller 16 inhibits gathering of the detected blood glucose level as the monitoring information on the basis of an interrupt instruction, as shown in FIG. 5. The data of the blood glucose level continues being sent from the blood glucose meter 14 to the controller 16. The controller 16 may generate an interrupt instruction to inhibit the gathering of the monitoring information at the same time as transmitting a control signal to the insulin pump 12. Prohibiting the gathering of the monitoring information refers to, for example, destroying the received data of the blood glucose level and not storing the data in the internal memory. The blood glucose meter 14 only has to repeat the cyclical operation and no change needs to be added to the processing operation of the blood glucose meter 14. Also in this case, similarly to the foregoing case, the local change in the blood glucose level is not gathered as the monitoring information. Therefore, even if the medication site and the site where the blood glucose level is detected are close to each other, the medication can be carried out on the basis of the correct blood glucose level.

### (4) Operation of Medication System According to Third Embodiment

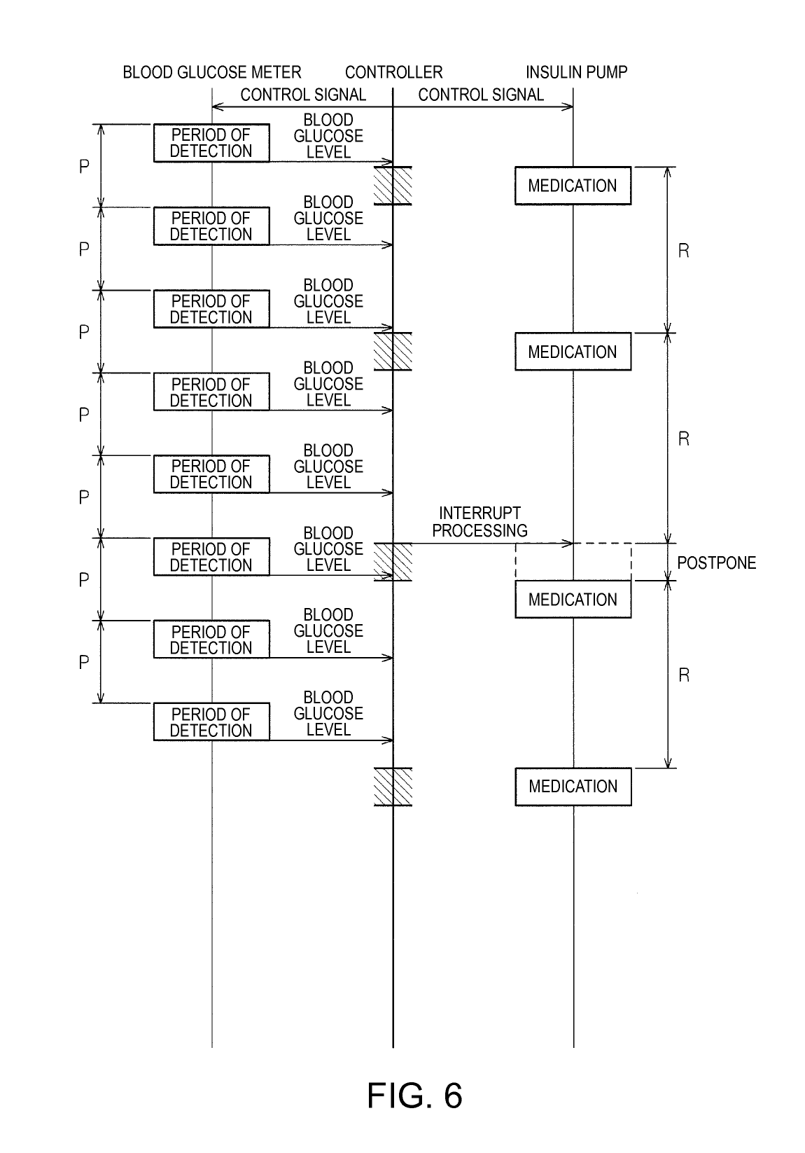

In a third embodiment, if the period of detection of the blood glucose level and the detection inhibiting period coincide with each other, the controller 16 postpones the medication instruction step until the period of detection of the biological information ends on the basis of an interrupt instruction, as shown in FIG. 6. The data of the blood glucose level continues being sent from the blood glucose meter 14 to the controller 16. The controller 16 may predict the detection inhibiting period on the basis of the cycle R of medication and transmit a control signal to the insulin pump 12 in such a way that the period of detection of the blood glucose level and the detection inhibiting period do not coincide with each other. The blood glucose meter 14 only has to repeat the cyclical operation and no change needs to be added to the processing operation of the blood glucose meter 14. Also in this case, similarly to the foregoing case, the local change in the blood glucose level is not gathered as the monitoring information. Therefore, even if the medication site and the site where the blood glucose level is detected are close to each other, the medication can be carried out on the basis of the correct blood glucose level.

### (5) Operation of Medication System According to Fourth Embodiment

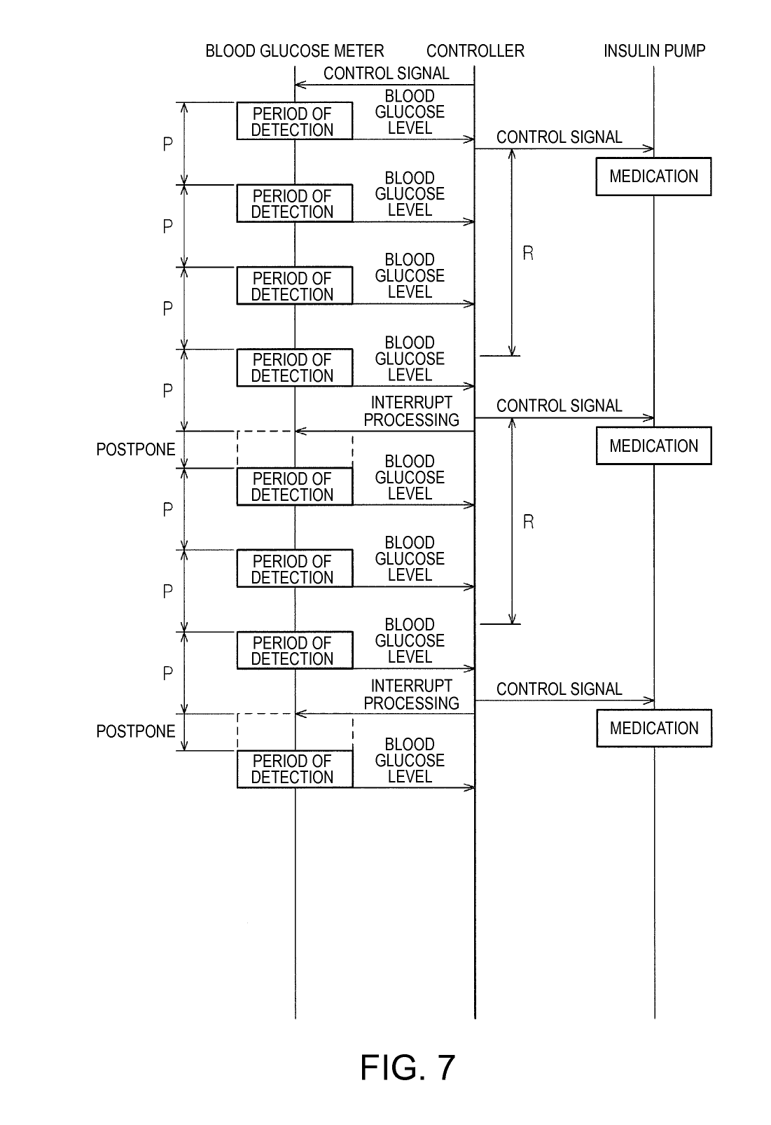

In a fourth embodiment, if the period of detection of the blood glucose level and the detection inhibiting period coincide with each other, the controller 16 postpones the medication instruction step until receiving the data of the detected blood glucose level, and postpones the biological information gathering instruction step until after receiving the data of the blood glucose level and after confirming that the medication instruction step is absent or after the lapse of the detection inhibiting period, on the basis of an interrupt instruction, as shown in FIG. 7. The controller 16 may predict the detection inhibiting period on the basis of the cycle R of medication and transmit a control signal to the blood glucose meter 14 in such a way that the period of detection of the blood glucose level and the detection inhibiting period do not coincide with each other. Since the medication is started after the data of the blood glucose level is received, and the detection of the blood glucose level is postponed until the medication is absent or until after the lapse of the detection inhibiting period, the coincidence of the period of detection of the blood glucose level and the detection inhibiting period can be securely avoided. Also in this case, similarly to the foregoing case, the local change in the blood glucose level is not gathered as the monitoring information. Therefore, even if the medication site and the site where the blood glucose level is detected are close to each other, the medication can be carried out on the basis of the correct blood glucose level.

While the embodiments are described in detail above, a person skilled in the art will readily understand that a number of modifications are possible without substantially departing from the new matters and advantageous effects of the invention. Therefore, all such modifications are included in the scope of the invention. For example, a term described at least once along with a different term with a broader meaning or the same meaning in the specification and drawings can be replaced with the different term at any point in the specification and drawings. Also, the configurations and operations of the insulin pump 12, the blood glucose meter 14 and the like are not limited to those described in the embodiments and various modifications can be made. Moreover, the functions of the controller 16 may be included in one of the insulin pump 12 and the blood glucose meter 14.

The entire disclosure of Japanese Patent Application No. 2014-236288 filed Nov. 21, 2014 is expressly incorporated by reference herein.

What is claimed is:

1. A medication control method comprising:
a medication instruction step in which an instruction to perform medication to a living body is given;
a biological information gathering instruction step in which an instruction to perform an operation of detecting biological information that can change due to the medication as a factor and gathering the biological information as monitoring information is given; and
an instruction step in which one of the medication instruction step and the biological information gathering instruction step is inhibited or postponed if a period of detection of the biological information carried out according to the biological information gathering instruction step and a period during which the medication carried out according to the medication instruction step influences the detection of the biological information coincide with each other.

2. The medication control method according to claim 1, wherein
a period during which an interrupt instruction is given is defined as the period during which the medication carried out according to the medication instruction step has influence, and a start of the period during which the interrupt instruction is given with respect to a medication start timing and an end of the period during which the interrupt instruction is given with respect to a medication end timing are set.

3. The medication control method according to claim 1, wherein
in the instruction step, the detection of the biological information is inhibited.

4. The medication control method according to claim 1, wherein
in the instruction step, gathering the biological information detected according to the biological information gathering instruction step, as the monitoring information, is inhibited.

5. The medication control method according to claim 1, wherein
in the interrupt instruction step, the medication instruction step is postponed until the period of detection of the biological information ends.

6. The medication control method according to claim 1, wherein
in the instruction step, the medication instruction step is postponed until transfer of the biological information that is detected is received, and the biological information gathering instruction step is postponed until after the transfer of the biological information is received and until after it is confirmed that the medication instruction step is absent or until after the lapse of the period during which the medication carried out according to the medication instruction step has influence.

7. A medication control program causing a computer to carry out:
a medication instruction procedure in which an instruction to perform medication to a living body is given;
a biological information gathering instruction procedure in which an instruction to perform an operation of detecting biological information that can change due to the medication as a factor and gathering the biological information as monitoring information is given; and
an instruction procedure in which one of the medication instruction procedure and the biological information gathering instruction procedure is inhibited or postponed if a period of detection of the biological information carried out according to the biological information gathering instruction procedure and a period during which the medication carried out according to the medication instruction procedure influences the detection of the biological information coincide with each other.

8. A medication system comprising:
a medication unit which performs medication to a living body;
a biological information gathering unit which detects biological information that can change due to the medication as a factor, and which gathers the biological information as monitoring information; and
a control unit which gives an instruction to perform the medication and to perform an operation of gathering the monitoring information, and carries out processing to inhibit or postpone the instruction to perform the medication and the detection of the biological information if a period of detection of the biological information and a period during which the medication influences the detection of the biological information coincide with each other.
